(19) United States
(12) Patent Application Publication
(10) Pub. No.: US 2023/0211047 A1
(43) Pub. Date: Jul. 6, 2023

(54) DENTAL BONE PLUG

(71) Applicant: Zimmer Biomet Spine, Inc., Westminster, CO (US)

(72) Inventors: Kaushal Kumar, Palm Beach Gardens, FL (US); Stefan Berger, Palm Beach Gardens, FL (US)

(21) Appl. No.: 18/091,130

(22) Filed: Dec. 29, 2022

Related U.S. Application Data
(60) Provisional application No. 63/295,767, filed on Dec. 31, 2021.

(51) Int. Cl. A61L 27/22; G06T 15/00; A61L 27/36

(52) U.S. Cl. CPC A61L 27/227; G06T 15/00; A61L 27/3608; A61L 27/3687; A61L 27/3691; A61L 27/365

(57) ABSTRACT

A method and system for making a bone plug using cortical bone material. A patient jaw having insufficient bone at a surgical site may be scanned to provide a 3D image which may be used to design a virtual bone plug and to fabricate the bone plug for placement within the patient. The bone plug may be formed from cortical bone that can be reconstituted and demineralized or demineralized and milled to shape.

### CROSS-REFERENCE TO RELATED APPLICATIONS

This application claims the benefit of U.S. Provisional Application No. 63/295,767, titled “DENTAL PLUG” and filed on Dec. 31, 2021, which application is incorporated herein by reference in its entirety.

### TECHNICAL FIELD

The present disclosure relates to a bone void plug, the preparation of the bone void plug, and use of the bone void plugs, e.g., in promoting bone growth.

### BACKGROUND

In the fields of orthopedics (e.g., reconstructive, trauma, spine, and dentistry), effective repair of bone defects, which may have been caused by disease, injury, wounds, or surgery, has long been a goal. A number of materials and compositions have been considered, evaluated, or used. Beyond their ability to promote bone growth, the biological, physical, and mechanical properties of the materials and compositions are factors, among others, affecting their suitability and performance in various applications.

Bone grafting has been commonly used to augment healing in treating a variety of musculoskeletal disorders. Grafting techniques in this field have been practiced for over 100 years and include procedures employing allograft and/or xenograft materials. Conventional cancellous bone provides both a natural tissue scaffold and osteoinductive growth factors and may also contain osteogenic components (e.g., mesenchymal stem cells) if obtained with bone marrow. Allogenic cancellous bone, however, is not acceptable or otherwise available for all patients. Autograft sources, as well as allograft sources, are relatively limited, may be cost prohibitive, and/or may be painful to obtain.

### SUMMARY

The present invention provides bone void plugs, methods to prepare bone void plugs, and uses of bone void plugs for example, to promote bone growth. For instance, in one example, the present invention includes three-dimensional (3D) porous bone void plugs that are formed from a reconstituted cortical bone graft, as described herein. In another embodiment, the bone plug may be formed from artificial or synthetic bone having characteristics of cortical bone.

As compared to bone void filler compositions in the form of a paste or putty, the bone void plug of the present disclosure is volume stable. That is, the shape of the bone void plug is substantially maintained after hydration. Maintaining the shape is important during bone augmentation to regenerate sufficient bone that can eventually receive a dental implant. In one example, a volume of the bone void plug after hydration is within about five percent (5) of the pre-hydration volume.

The bone void filler may be compressible such that the dimensional shape and volume can be altered, e.g., by compression, but is capable of retaining the same shape and volume after the force that altered the shape and volume is removed.

In one example, the bone void plug can comprise cortical bone as it can be advantageous to incorporate the strength characteristics of cortical bone into the bone void. The 3D cortical bone void plug can be harvested from human bones, non-human bones, and/or may be artificial or synthetic bone. The cortical bone can be 3D milled or reconstituted and can be demineralized to expose a desired percentage or proportion of bone morphogenetic proteins (BMP), which may be used to, for example, promote bone growth in the bone void.

As discussed herein, the shape, porosity, pore diameter, etc., can be designed based on the patient's and indication needs. According to one aspect of the present invention a method may be provided for augmenting bone with the 3D bone void plus in a patient having inadequate bone quality or quantity, the method comprising the steps of: obtaining a virtual 3D image of an area of bone to be augmented, determining the area of bone to be augmented including a virtual representation of a bone void, developing a 3D virtual representation of the bone void plug to be placed in the bone void, determine various features of the 3D dental plug, determine dimensional information and characteristic information of the 3D dental plug, and sending the information to a manufacturer.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

These and other examples, advantages, and features of the present dental membranes will be set forth in part in the following Detailed Description and the accompanying drawings. This Overview is intended to provide non-limiting examples of the present subject matter—it is not intended to provide an exclusive or exhaustive explanation. The Detailed Description and drawings are included to provide further information about the present porous metal dental implants.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 illustrates a perspective view of a jaw with insufficient bone for implant placement;

FIG. 2 illustrates a cross-sectional view of the jaw including a bone plug according to at least one embodiment of the present disclosure;

FIG. 3 illustrates a top-down view of a patient's jaw having more than one bone void according to at least one embodiment of the present disclosure;

FIG. 4 illustrates a perspective view of a bone plug according to at least one embodiment of the present disclosure

FIG. 5 illustrates a perspective view of a bone plug according to at least one embodiment of the present disclosure;

FIG. 6 illustrates a perspective view of a bone plug according to at least one embodiment of the present disclosure;

FIG. 7 illustrates a perspective view of a bone plug according to at least one embodiment of the present disclosure

FIG. 8 illustrates a perspective view of a bone plug according to at least one embodiment of the present disclosure;

FIG. 9 illustrates a patient-specific bone plug according to at least one embodiment of the present disclosure;

FIG. 10 illustrates a flow diagram of forming a bone plug according to at least one embodiment of the present disclosure; and

FIG. 11 illustrates an example system according to at least one embodiment of the present disclosure.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The present invention relates generally to 3D bone void plugs, design and manufacture of the 3D bone void plugs, and the use of the 3D bone void plugs in preventing or inhibiting bone loss and/or promoting bone growth.

The 3D bone void plugs (also referred to herein as “plugs”) can be formed from one or more materials (including to promote mineralization) including, but not limited to, autograft, allograft or xenograft bone (e.g., cortical, corticocancellous chips, scaffolds), synthetic or artificial bone (e.g., bioceramics), biocomposites, proteins, lipids, peptides, and polymers. Some exemplary materials for the 3D bone void plugs are discussed in U.S. Pat. No. 8,613,938, which is incorporated by reference in its entirety.

In one embodiment, the 3D bone void plug is in the form of a scaffold. The scaffold restores function and/or regenerates bone by providing a temporary matrix for cell proliferation and extracellular matrix deposition with consequent bone in-growth until new bony tissue is restored and/or regenerated. The matrix may also provide a template for vascularization of this tissue. The scaffold may actively participate in the regenerative process through the release of growth factors, minerals and/or other substances beneficial to the bone formation process if such are present in the scaffold.

The macro and micro-structural properties of the scaffold influence the survival, signaling, growth, propagation, and reorganization of cells. They may also influence cellular gene expression and phenotype preservation. The following scaffold characteristics contribute to bone formation: cell biocompatibility; surface chemistry; biodegradability, porosity; and/or pore size.

Various manufacturing methods can form the bone plug. For example, additive and/or subtractive manufacturing methods can be used with bone materials to form the bone plug. This flexibility in manufacturing allows a user to design a patient specific plug that can best help regenerate bone based on the patient's needs. Further, the bone plug can be at least partially demineralized or fully demineralized.

As discussed herein, the bone plug can advantageously be volume stable. That is, the bone plug, after implantation, does not lose more than less than about 5% of a total volume. Maintaining the shape and integrity of the bone plug after implantation is important to successfully regenerate a bone void.

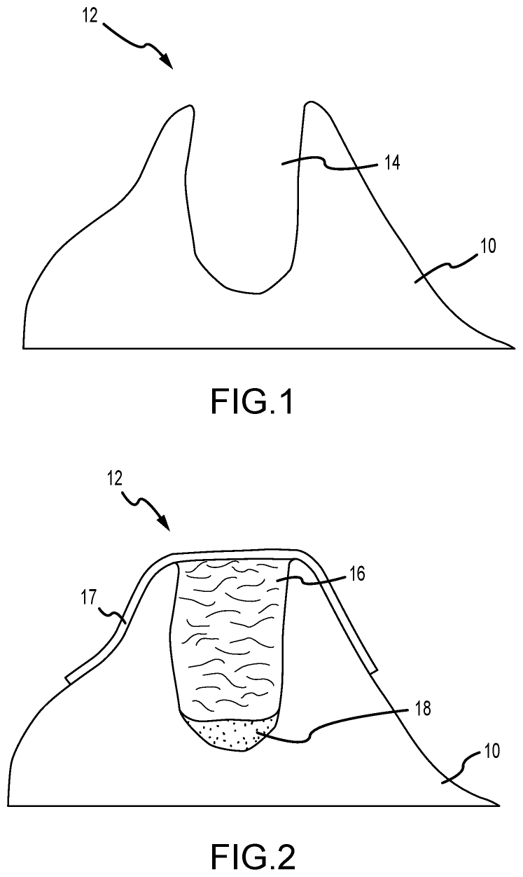

FIG. 1 shows a patient jaw 10 having insufficient bone at a surgical site 12 including a bone void 14. The insufficient bone in the patient jaw 10 may be due to factors such as periodontal disease, lost teeth, bone resorption, a thin maxillary and mandibular arch due to genetics, etc. The surgical site 12 can vary between patient's and can have a variety of shapes and sizes. During bone regeneration to form a dental ridge sufficient to receive a dental implant, the shape and size of the regenerated bone is important. A bone void surrounded by four walls may be easier to contain the bone void plug as compared to a bone void only having two or the four walls. As described herein, a 3D bone void plug 16 can be received within the bone void 14 of the patient. The bone plug 16 can be secured in the bone void 14 using, for example, screws, tacks, adhesion, sutures, etc.

FIG. 2 illustrates a cross-sectional view of a bone plug 16 within a bone void 14 of the patient. In some examples, the bone plug 16 can be manufactured in stock shapes and sizes and a user can modify the bone plug 16 during implantation to create a size and shape that best fits the patient. As seen in FIG. 2, along with the plug 16, bone graft material 18 can also be used to fill in gaps that the bone plug 16 does not engage with. Once the bone plug 16 is inserted, a dental membrane 17 can be secured to the patient and the soft tissue (not shown) can be sutured together for healing.

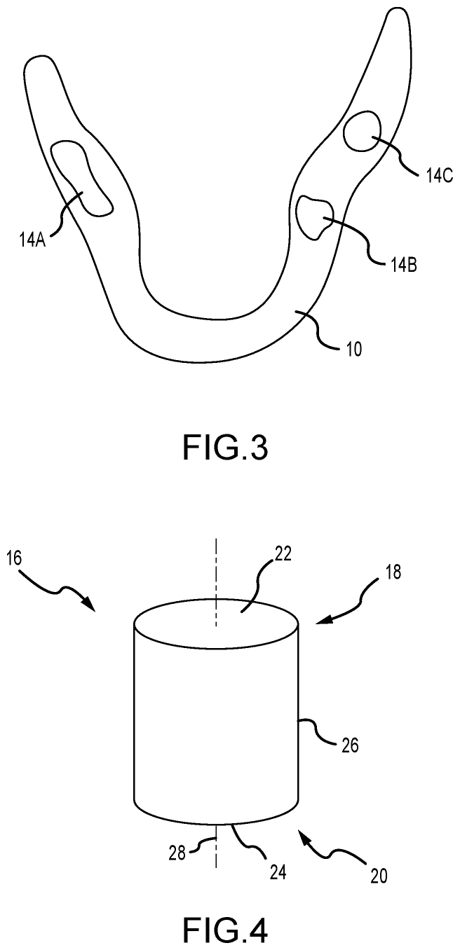

FIG. 3 illustrates a top-down view of a patient's jaw 10 having more than one bone void 14A-14C. In the illustrated embodiment, bone voids 14A and 14B are the shape and size of naturally occurring bone void and bone void 14C is a modified bone void. It will be appreciated that the patient may have any number of bone void(s) and that each bone void may be any shape or size. In some embodiments, the bone void may be modified such as modified bone void 14C. Such modified bone void may be modified by a surgeon such that the shape of the bone plug 16 substantially matches the shape of the bone void 14C. Therefore, a surgeon can modify one or both of the bone plug 16 and the bone void 14 to ensure that the bone plug 16 fits securely and fills up the bone void 14C (or any bone void) as much as possible.

Turning to FIG. 4, a bone plug 16 is illustrated. The bone plug 16 includes a body 26 extending from a first end 18 to a second end 20. The first end 18 includes a top surface 22 that generally will be a top surface of a bone void such as the bone void 14A, 14B, 14C. The body 26 also includes a bottom surface 24 opposite the top surface 22. The bone plug 16 is a porous structure. While the illustrated bone plug 16 is shown as a cylinder and having a circular cross-section, it will be appreciated that in other embodiments the bone plug 16 may be any other shape and/or size. Further, the bone plug 16 can be provided to users in a standard stock shape, e.g., but not limited to, a cylinder, square, triangle, but can be modified prior to or during a surgical procedure to better match the bone void shape of the patient. Additionally cross-sectional shapes of the bone plug 16 are not limited. The cross-sectional shape can be any shape. As discussed herein, the porosity of the bone plug 16 can be consistent or can vary. In one example, the porosity of the bone plug 16 increases along the longitudinal axis 28. That is, the porosity of the first end 18 (e.g., a coronal end) is less than the porosity of the second end 20 (e.g., an apical end).

As previously described, the bone plug 16 can be formed from cortical bone. In some embodiments, the bone plug 16 can be formed from reconstituted cortical bone. The cortical bone may be reconstituted so as to reform the cortical bone into a desired shape. For example, the cortical bone may be reconstituted into a shape to fit inside of the bone void 14A, 14B, 14C. The cortical bone can be reconstituted using, for example, 3D printing, chemical cross-linking, and/or sintering of bio-ceramics. In such embodiments, the bone plug 16 (comprising reconstituted cortical bone) may be demineralized after the bone plug 16 is reconstituted. Demineralizing the cortical bone advantageously exposes the BMP of cortical bone that is absent or exists in lower concentrations from other types of bone (e.g., cancellous). Thus, demineralization enables the cortical bone to provide BMP, which may promote bone growth in the bone void 14A, 14B, 14C. The demineralization of the bone plug 16 may be controlled to as to expose a desired amount of BMP. Additionally or alternatively, the demineralization of the bone plug 16 may be controlled to balance the BMP exposure and mechanical properties of the bone plug 16 as demineralization may result in lower mechanical properties of the bone plug 16. In at least one example, the bone plug 16 can be demineralized so as to expose 25% BMP, 60% BMP, or any other percentage of BMP. Further, various portions of the bone plug 16 can be demineralized at different rates. For example, a first portion of the bone plug 16 may have a BMP percentage that is different than a second portion of the bone plug 16. The bone plug 16 may be demineralized using one or more processes such as: grinding the cortical bone, demineralizing the cortical bone with an acid solution, washing the cortical bone with water or a phosphate buffered solution, and/or washing the cortical bone with ethanol and drying the cortical bone. Demineralized cortical bone may be obtained from a source such as a commercial bone or tissue bank. In other embodiments, the cortical bone may be demineralized (without reconstituting) and further milled or shaped to a desired shape. In such embodiments, the demineralized cortical bone may be of a composition in which the cortical bone can be milled

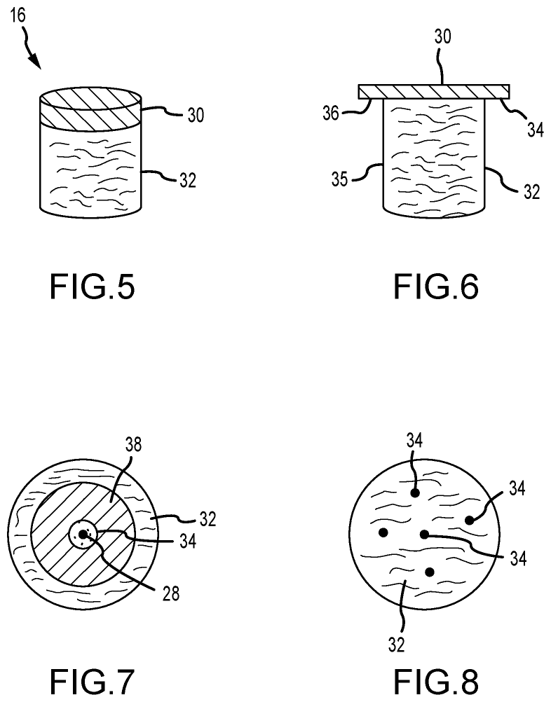

FIGS. 5-8 illustrate various bone void plug example shapes and sizes. For example, FIG. 5 illustrates the bone plug 16 having a first portion 30 and a second portion 32. The first portion 30 and the second portion 32 may have, for example, different BMP percentages. In other instances, the first portion 30 and the second portion 32 may have the same BMP percentage. As discussed herein, the bone plug 16 can be harvested from human and/or non-human bone. FIG. 6 illustrates a bone plug 16 including an annular flange 34 and a body 35. The flange 34 includes a shoulder surface 36 that is configured to engage a portion of the dental ridge of the patient. The shoulder may help maintain the position of the bone plug 16 when inserted into the bone void. FIG. 7 illustrates an example where the BMP percentage changes axially. That is, a center of the bone plug 16 along the longitudinal axis 28 has the lowest BMP exposed. The outer portion can have the highest BMP exposed. Further, the bone plug 16 can have a transition layer such that the BMP exposure changes from the lowest BMP exposed to the lowest BMP exposed as you move axially from the longitudinal axis. FIG. 8 illustrates an example where the bone plug 16 includes ribs 34, formed from, e.g., sintering material, lasering, and are positioned throughout the plug 16 to help with the integrity of the plug 16.

It will be appreciated that the examples provided above are example shapes and sizes, and other embodiments including different porosities, different materials, sizes and shapes are contemplated.

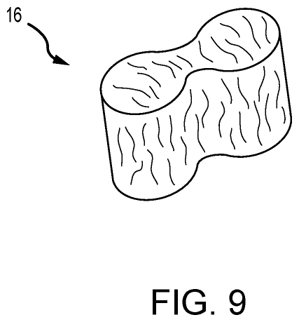

In some embodiments, the bone plug 16 can be modified and/or manufactured to custom fit a bone void such as the bone void 14A, 14B, 14C, as discussed herein. An example custom fit bone void 16 using a method 1000 described below is shown in FIG. 9.

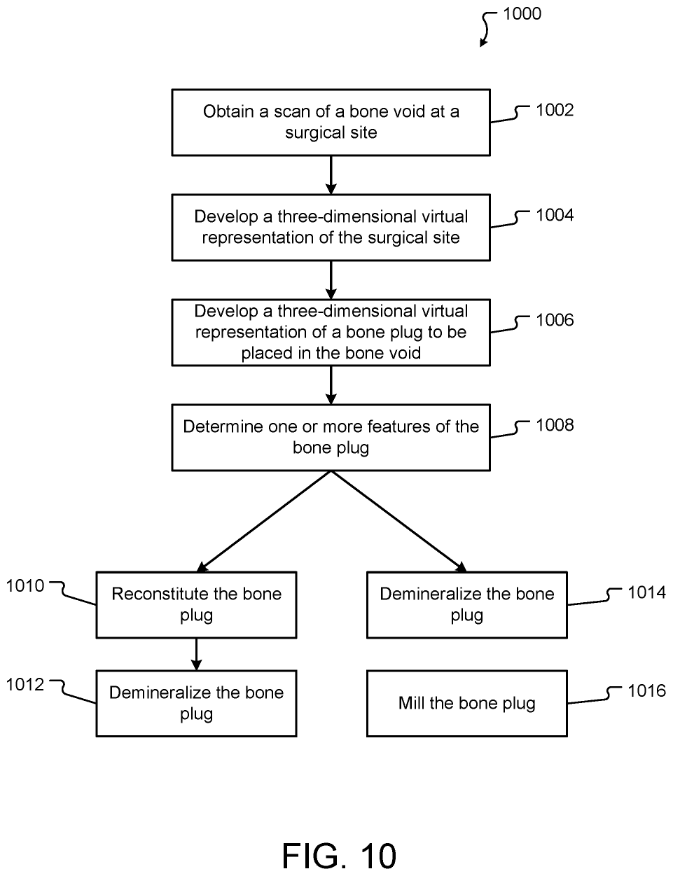

Turning to FIG. 10, a method 1000 of designing a bone plug 16 is illustrated. Any step and any combination of steps of the method 1000 may be executed manually by a user, automatically by a computing device, and/or semi-automatically by a user and/or a computing device, manufacturing machine, or the like.

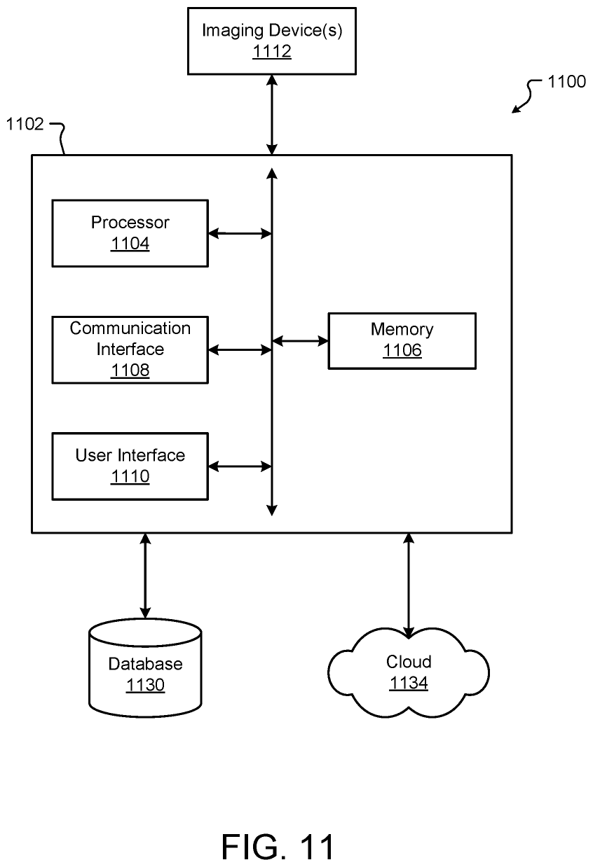

At step 1002, the method 1000 can include obtaining a scan of a patient's jaw including bone void(s) such as the bone voids 14A, 14B, 14C. The scan may be received from an imaging device such as an imaging device 1112 (shown in FIG. 11) of a system such as a system 1100. The scan may be a computerized tomography (CT) scan, Cone Beam Computed Tomography (CBCT) scan, Magnetic Resonance Imaging (MRI) scan and/or intra-oral scan, said scan may include a surface topography of the surgical site. Herein, the scan may be obtained from a scanning step performed by a user such as a clinician. Alternatively, the clinician may obtain scan data from a previous scanning operation. If a CBCT scan and/or a CT scan is used, the exact bone quality, region of graft, and amount of graft required may be determined using the CBCT scan and/or the CT scan.

At step 1004, the scan data is received, and a 3D virtual representation of the patient can be developed by a processor such as a processor 1106 of a system such as the system 1100. The 3D virtual representation of the patient may be displayed on, for example, a user interface such as a user interface 1110.

At step 1006, a 3D virtual representation of the bone plug to be placed into the bone void can be generated based on the data such as, for example, dimensional information of the bone void in the patient's jaw. The 3D virtual representation of the bone plug may be generated by, for example, the processor 1106.

The method 1000 can include at step 1008 determining various features of the bone plug. That is, the porosity, types of bone, any additional securement features, etc., can be designed onto the virtual representation of the bone plug. The various features may be determined automatically by, for example, the processor. In other embodiments the various features may be determined manually by a user such as a clinician or dental surgeon. In still other embodiments, the various features may be determined by a combination of user input and features determined automatically by the processor. In further embodiments, the features may be determined automatically by the processor and the user may approve or change the features. After the shape and various features of the bone void plug are determined, manufacturing information can be developed and sent to a manufacturer for fabrication.

The method 1000 can also include at step 1010 reconstituting the bone plug. In such steps, the bone plug may comprise cortical bone. The cortical bone may be reconstituted so as to reform the cortical bone into a desired shape. For example, the cortical bone may be reconstituted into a shape to fit inside of the bone void.

The method 1000 can also include at step 1012 demineralizing the reconstituted bone plug (which may have been reconstituted in, for example, the step 1010 described above). As previously described, demineralizing the cortical bone advantageously exposes the BMP of cortical bone that is absent from other types of bone (e.g., cancellous). Thus, demineralization enables the cortical bone to expose BMP, which may promote bone growth in the bone void. The demineralization of the bone plug may be controlled to as to expose a desired amount of BMP. For example, the bone plug can be demineralized so as to expose 25% BMP, 60% BMP, or any other percentage of BMP. Further, various portions of the bone plug can be demineralized at different rates. For example, a first portion of the bone plug may have a BMP percentage that is different than a second portion of the bone plug. The bone plug may be demineralized using one or more processes such as: grinding the cortical bone, demineralizing the cortical bone with an acid solution, washing the cortical bone with water or a phosphate buffered solution, and/or washing the cortical bone with ethanol and drying the cortical bone. Demineralized cortical bone may be obtained from a source such as a commercial bone or tissue bank. In other embodiments, the cortical bone may be milled or shaped to a desired shape, then demineralized (without reconstituting).

Alternatively after the step 1008, the method 1000 may include at step 1014 demineralizing the bone plug. The step 1014 may be the same as or similar to the step 1012 except that the bone plug is not reconstituted.

The method 1000 may also include after the step 1014, milling the demineralized bone plug. The bone plug may be milled manually or automatically using for example, a milling machine, a lathe, and/or a CNC machine. The demineralized bone may be milled to a custom shape to fit, for example, the bone void.

The present disclosure encompasses embodiments of the method 1000 that comprise more or fewer steps than those described above, and/or one or more steps that are different than the steps described above.

As noted above, the present disclosure encompasses methods with fewer than all of the steps identified in FIG. 10 (and the corresponding description of the method 1000), as well as methods that include additional steps beyond those identified in FIG. 10 (and the corresponding description of the method 1000). The present disclosure also encompasses methods that comprise one or more steps from one method described herein, and one or more steps from another method described herein. Any correlation described herein may be or comprise a registration or any other correlation.

Having described a method of 1000 of making a custom bone void plug, the present disclosure also includes a system 1100that may be employed in accordance with at least some of the example embodiments herein. Although various embodiments may be described herein in terms of this exemplary computer system, after reading this description, it may become apparent to a person skilled in the relevant art(s) how to implement the disclosure using other computer systems and/or architectures.

Turning first to FIG. 11, a block diagram of a system 1100 according to at least one embodiment of the present disclosure is shown. The system 1100 may be used to design and/or form a bone plug such as the bone plug 16 and/or carry out one or more other aspects of one or more of the methods disclosed herein. The system 1100 comprises a computing device 1102, one or more imaging devices 1112, a database 1130, and/or a cloud or other network 1134. Systems according to other embodiments of the present disclosure may comprise more or fewer components than the system 1100. For example, the system 1100 may not include the imaging device 1112, one or more components of the computing device 1102, the database 1130, and/or the cloud 1134.

The computing device 1102 comprises a processor 1104, a memory 106, a communication interface 1108, and a user interface 110. Computing devices according to other embodiments of the present disclosure may comprise more or fewer components than the computing device 1102.

The processor 1104 of the computing device 1102 may be any processor described herein or any similar processor. The processor 1104 may be configured to execute instructions stored in the memory 1106, which instructions may cause the processor 1104 to carry out one or more computing steps utilizing or based on data received from the imaging device 1112, the database 1130, and/or the cloud 1134.

The memory 1106 may be or comprise RAM, DRAM, SDRAM, other solid-state memory, any memory described herein, or any other tangible, non-transitory memory for storing computer-readable data and/or instructions. The memory 1106 may store information or data useful for completing, for example, any step of the method 1000 described herein, or of any other methods. Such content, if provided as in instruction, may, in some embodiments, be organized into one or more applications, modules, packages, layers, or engines. Alternatively or additionally, the memory 1106 may store other types of content or data (e.g., machine learning models, artificial neural networks, deep neural networks, etc.) that can be processed by the processor 1104 to carry out the various method and features described herein. Thus, although various contents of memory 1106 may be described as instructions, it should be appreciated that functionality described herein can be achieved through use of instructions, algorithms, and/or machine learning models. The data, algorithms, and/or instructions may cause the processor 1104 to manipulate data stored in the memory 1106 and/or received from or via the imaging device 1112, the database 1130, and/or the cloud 1134.

The computing device 1102 may also comprise a communication interface 1108. The communication interface 1108 may be used for receiving image data or other information from an external source (such as the imaging device 1112, the database 1130, the cloud 1134, and/or any other system or component not part of the system 1100), and/or for transmitting instructions, images, or other information to an external system or device (e.g., another computing device 1102, the imaging device 1112, the database 1130, the cloud 1134, and/or any other system or component not part of the system 1100). The communication interface 1108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11ac/ax/a/b/g/n, Bluetooth, NFC, ZigBee, and so forth). In some embodiments, the communication interface 1108 may be useful for enabling the device 1102 to communicate with one or more other processors 1104 or computing devices 1102, whether to reduce the time needed to accomplish a computing-intensive task or for any other reason.

The computing device 1102 may also comprise one or more user interfaces 1110. The user interface 1110 may be or comprise a keyboard, mouse, trackball, monitor, television, screen, touchscreen, and/or any other device for receiving information from a user and/or for providing information to a user. The user interface 1110 may be used, for example, to receive a user selection or other user input regarding any step of any method described herein. Notwithstanding the foregoing, any required input for any step of any method described herein may be generated automatically by the system 1100 (e.g., by the processor 1104 or another component of the system 1100) or received by the system 1100 from a source external to the system 1100. In some embodiments, the user interface 1110 may be useful to allow a dental surgeon or other user to modify instructions to be executed by the processor 1104 according to one or more embodiments of the present disclosure, and/or to modify or adjust a setting of other information displayed on the user interface 1110 or corresponding thereto.

Although the user interface 1110 is shown as part of the computing device 1102, in some embodiments, the computing device 1102 may utilize a user interface 1110 that is housed separately from one or more remaining components of the computing device 1102. In some embodiments, the user interface 1110 may be located proximate one or more other components of the computing device 1102, while in other embodiments, the user interface 1110 may be located remotely from one or more other components of the computer device 1102.

The imaging device 1112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 1112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 1112 may be used to obtain first image data (e.g., a first image) at a first time, and a second imaging device 1112 may be used to obtain second image data (e.g., a second image) at a second time after the first time. The imaging device 1112 may be capable of taking a 2D image or a 3D image to yield the image data. The imaging device 1112 may be or comprise, for example, an ultrasound scanner (which may comprise, for example, a physically separate transducer and receiver, or a single ultrasound transceiver), a scanner such as CBCT, Mill and/or intra-oral scanner for obtaining 3D images of a dental cavity, an X-ray imaging device, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 1112 suitable for obtaining images of an anatomical feature of a patient. The imaging device 1112 may be contained entirely within a single housing, or may comprise a transmitter/emitter and a receiver/detector that are in separate housings or are otherwise physically separated.

The database 1130 may store information about a patient's anatomy at and/or proximate the surgical site, for use by a user of the computing device 1102 or of the system 1100; one or more images useful in connection with a surgery to be completed by or with the assistance of one or more other components of the system 1100; and/or any other useful information. The database 1130 may be configured to provide any such information to the computing device 1102 or to any other device of the system 1100 or external to the system 1100, whether directly or via the cloud 1134.

The cloud 1134 may be or represent the Internet or any other wide area network. The computing device 1102 may be connected to the cloud 1134 via the communication interface 1108, using a wired connection, a wireless connection, or both. In some embodiments, the computing device 1102 may communicate with the database 1130 and/or an external device (e.g., a computing device) via the cloud 1134.

The system 1100 or similar systems may be used, for example, to carry out one or more aspects of any of the method 11000 described herein. The system 1100 or similar systems may also be used for other purposes.

In one or more examples, the described methods, processes, and techniques may be implemented in hardware, software, firmware, or any combination thereof If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

### EXAMPLES

To illustrate the dental plug, a non-limiting list of examples is provided here:

In Example 1, a method of forming a bone plug comprising at least one of milling or reconstituting a bone plug to a desired shape to yield a reconstituted bone plug, the bone plug comprising bone; and demineralizing the reconstituted bone plug until a desired percentage of bone morphogenetic proteins (BMP) is exposed in the reconstituted bone plug.

Example 2 can include, or can optionally be combined with the subject matter of Example 1, to optionally include wherein the bone is cortical bone and comprises at least one of artificial bone, allograft bone, or xenograft bone.

Example 3 can include, or can optionally be combined with the subject matter of one or any combination of Examples 1 or 2 to optionally include wherein the bone plug is a patient specific bone plug.

Example 4 can include, or can optionally be combined with the subject matter of one or any combination of Examples 1 through 3 to optionally include obtaining a virtual 3D image of a patient including a bone void at a surgical site; determining dimensions of the bone void of the patient from the virtual 3D image; developing a 3D virtual representation of a bone plug; and determining features of the virtual representation of the bone plug corresponding to the desired shape.

Example 5 can include, or can optionally be combined with the subject matter of one or any combination of Examples 1 through 4 to optionally include wherein the virtual 3D image of the surgical site is obtained from a CBCT, MRI and/or intraoral scan.

Example 6 can include, or can optionally be combined with the subject matter of one or any combination of Examples 1 through 5 to optionally include wherein the desired percentage of the BMP exposed is less than or equal to about 8%.

Example 7 can include, or can optionally be combined with the subject matter of one or any combination of Examples 1 through 6 to optionally include wherein the reconstituted bone plug is demineralized using at least one of an acid solution, a phosphate buffered solution, an ethanol solution.

Example 8 can include, or can optionally be combined with the subject matter of one or any combination of Examples 1 through 7 to optionally include wherein a first portion of the reconstituted bone plug is demineralized to expose a first desired percentage of BMP and a second portion of the reconstituted bone plug is demineralized to expose a second desired percentage of BMP.

In Example 9, a method of forming a bone plug comprising milling the bone plug to a desired shape; and demineralizing a bone plug until a desired percentage of bone morphogenetic proteins (BMP) is exposed, the bone plug comprising cortical bone.

Example 10 can include, or can optionally be combined with the subject matter of Example 9, to optionally include obtaining a virtual 3D image of a patient including a bone void at a surgical site; determining dimensions of the bone void of the patient from the virtual 3D image; developing a 3D virtual representation of a bone plug; and determining features of the virtual representation of the bone plug corresponding to the desired shape.

Example 11 can include, or can optionally be combined with the subject matter of one or any combination of Examples 9 or 10 to optionally include wherein the virtual 3D image of the surgical site is obtained from a CBCT, MRI and/or intraoral scan.

Example 12 can include, or can optionally be combined with the subject matter of one or any combination of Examples 9 through 11 to optionally include wherein the desired percentage of the BMP exposed is less than or equal to about 8%.

Example 13 can include, or can optionally be combined with the subject matter of one or any combination of Examples 9 through 12 to optionally include wherein the bone plug is demineralized using at least one of an acid solution, a phosphate buffered solution, an ethanol solution.

Example 14 can include, or can optionally be combined with the subject matter of one or any combination of Examples 9 through 13 to optionally include wherein a first portion of the bone plug is demineralized to expose a first desired percentage of BMP and a second portion of the bone plug is demineralized to expose a second desired percentage of BMP.

In Example 15, a bone plug comprising a body extending from a first end to a second end, the body shaped in a desired shape using at least one of reconstitution or milling, and the body having a desired percentage of BMP exposed by demineralization.

Example 16 can include, or can optionally be combined with the subject matter of Example 15, to optionally include wherein the body comprises cortical bone.

Example 17 can include, or can optionally be combined with the subject matter of one or any combination of Examples 15 or 16 to optionally include wherein the cortical bone comprises at least one of allograft and xenograft.

Example 18 can include, or can optionally be combined with the subject matter of one or any combination of Examples 15 through 17 to optionally include wherein the bone plug is a patient specific bone plug.

Example 19 can include, or can optionally be combined with the subject matter of one or any combination of Examples 15 through 18 to optionally include wherein the desired percentage of the BMP exposed is less than or equal to about 8%.

Example 20 can include, or can optionally be combined with the subject matter of one or any combination of Examples 15 through 19 to optionally include wherein a first portion of the body is demineralized to expose a first desired percentage of BMP and a second portion of the body is demineralized to expose a second desired percentage of BMP.

What is claimed is:

1. A method of forming a bone plug comprising:
at least one of milling or reconstituting a bone plug to a desired shape to yield a reconstituted bone plug, the bone plug comprising bone; and
demineralizing the reconstituted bone plug until a desired percentage of bone morphogenetic proteins (BMP) is exposed in the reconstituted bone plug.

2. The method of claim 1, wherein the bone is cortical bone and comprises at least one of artificial bone, allograft bone, or xenograft bone.

3. The method of claim 1, wherein the bone plug is a patient specific bone plug.

4. The method of claim 1, further comprising:
obtaining a virtual 3D image of a patient including a bone void at a surgical site;
determining dimensions of the bone void of the patient from the virtual 3D image;
developing a 3D virtual representation of a bone plug; and
determining features of the virtual representation of the bone plug corresponding to the desired shape.

5. The method of claim 4, wherein the virtual 3D image of the surgical site is obtained from a CBCT, MRI and/or intraoral scan.

6. The method of claim 1, wherein the desired percentage of the BMP exposed is less than or equal to about 8%.

7. The method of claim 1, wherein the reconstituted bone plug is demineralized using at least one of an acid solution, a phosphate buffered solution, an ethanol solution.

8. The method of claim 1, wherein a first portion of the reconstituted bone plug is demineralized to expose a first desired percentage of BMP and a second portion of the reconstituted bone plug is demineralized to expose a second desired percentage of BMP.

9. A method of forming a bone plug comprising:
milling the bone plug to a desired shape; and
demineralizing a bone plug until a desired percentage of bone morphogenetic proteins (BMP) is exposed, the bone plug comprising cortical bone.

10. The method of claim 9, further comprising:
obtaining a virtual 3D image of a patient including a bone void at a surgical site;
determining dimensions of the bone void of the patient from the virtual 3D image;
developing a 3D virtual representation of a bone plug; and
determining features of the virtual representation of the bone plug corresponding to the desired shape.

11. The method of claim 10, wherein the virtual 3D image of the surgical site is obtained from a CBCT, MM and/or intraoral scan.

12. The method of claim 9, wherein the desired percentage of the BMP exposed is less than or equal to about 8%.

13. The method of claim 9, wherein the bone plug is demineralized using at least one of an acid solution, a phosphate buffered solution, an ethanol solution.

14. The method of claim 9, wherein a first portion of the bone plug is demineralized to expose a first desired percentage of BMP and a second portion of the bone plug is demineralized to expose a second desired percentage of BMP.

15. A bone plug comprising:
a body extending from a first end to a second end, the body shaped in a desired shape using at least one of reconstitution or milling, and the body having a desired percentage of BMP exposed by demineralization.

16. The bone plug of claim 15, wherein the body comprises cortical bone.

17. The bone plug of claim 16, wherein the cortical bone comprises at least one of allograft and xenograft.

18. The bone plug of claim 15, wherein the bone plug is a patient specific bone plug.

19. The bone plug of claim 15, wherein the desired percentage of the BMP exposed is less than or equal to about 8%.

20. The bone plug of claim 15, wherein a first portion of the body is demineralized to expose a first desired percentage of BMP and a second portion of the body is demineralized to expose a second desired percentage of BMP.
